(19) United States
(12) Patent Application Publication
(10) Pub. No.: US 2026/0079102 A1
(43) Pub. Date: Mar. 19, 2026

(54) SPECTROPHOTOMETRIC MEASUREMENT OF WATER IN HYDROCARBONS VIA WATER DROPLET COUNTING

(71) Applicant: Saudi Arabian Oil Company, Dhahran (SA)

(72) Inventors: Brian Parrott, Thuwal (SA); Maha Nour, Thuwal (SA)

(73) Assignee: Saudi Arabian Oil Company, Dhahran (SA)

(21) Appl. No.: 18/888,595

(22) Filed: Sep. 18, 2024

(51) Int. Cl. G01N 21/31; G01N 21/85; G01N 33/28

(52) U.S. Cl. CPC G01N 21/31; G01N 21/85; G01N 33/2847; G01N 2201/06113

(57) ABSTRACT

A spectrophotometric system and method for quantifying water content in a liquid hydrocarbon stream is disclosed. The system can include at least one light source that produces a light having a desired wavelength and a flow chamber that allows passage of a sample of the liquid hydrocarbon stream. The light is configured to pass through the liquid hydrocarbon stream within the flow chamber. The system can also include at least one photosensitive detector array that measures an intensity of the light at one or more desired wavelengths of the light after it passes through the liquid hydrocarbon stream in the flow chamber. The intensity measurement of the at least one photosensitive detector array is used to quantify the undissolved water content of the liquid hydrocarbon stream.

### TECHNICAL FIELD

The present disclosure is generally related to a spectrophotometric system, and more specifically, a spectrophotometric system for quantifying water in a hydrocarbon composition.

### BACKGROUND

Hydrocarbon streams often contain contaminants that can create problems during processes involving the hydrocarbon streams, which reduce the quality and value of the products made from those processes. Water is one of the contaminants present in many hydrocarbon streams that can be a major concern. Water causes problems related to corrosion in the hydrocarbon processing facilities, and while operators can take measures to remove the water or protect assets from higher water levels, they need to know the level of water contamination to properly determine the appropriate method of handling the contamination. Most hydrocarbon-based products require a very low level of water to perform properly, and thus a relatively high quantity of water in the product can reduce the value of the product substantially. Thus, it is important to know the water content of the hydrocarbon stream for the purposes of knowing the potential quality of the downstream hydrocarbon product, and to provide the facility operator the opportunity to resolve any water contamination issues with the product in a dynamic fashion.

Free water in liquid hydrocarbon stream naturally separates from the other components due to gravity force in ambient conditions. However, measuring the free water (undissolved water) content in liquid hydrocarbons is challenging in common field conditions where there is high pressure, flowrate, and temperature, as well as potential turbulence. Under these conditions, the free water droplets are well mixed with the liquid hydrocarbons, making it hard to monitor water content, especially as bulk methods do not provide sufficient accuracy. Moreover, taking samples of the hydrocarbon stream and sending it to laboratories for analysis is not an optimal solution due to potential errors in sample collection (due to pressure changes, temperature changes, evaporation, etc.). Additionally, because it takes time to send the samples to a laboratory and to complete the analysis, the resulting laboratory data can be outdated by the time it is provided to a facilities operator, which makes it more difficult to utilize the measurements to effect meaningful change in the processing of the hydrocarbon stream.

Conventionally, water cut meters have sometimes been used for oil and gas applications. Water cut meters, in general, aim to measure the free water (above saturation) concentration in mixed hydrocarbon streams and are especially relevant when the quantity of water is fairly high. However, water cut meters are not accurate for hydrocarbon streams with relatively low concentrations of water (in ppm range), with significant errors appearing when the water cut is low enough that it is no longer bulk flow but widely separated droplets. Water cut measurements, in some cases, are capable of detecting as low as 5000-1000 ppm (0.5%-0.1%), but this leaves the required sensitivity too high for downstream applications that target low concentrations in the range of 1-1000 ppm. Accordingly, a significantly more sensitive technology is needed for these applications.

The present application addresses these and other challenges related to assessing the water content of hydrocarbon streams.

### SUMMARY

In a first aspect, a spectrophotometric system for quantifying water content in a liquid hydrocarbon stream is provided. The system includes at least one light source that produces a light having a desired wavelength, and a flow chamber configured to allow passage of a sample of the liquid hydrocarbon stream. The light is configured to pass through the liquid hydrocarbon stream within the chamber. The system also includes at least one photosensitive detector array configured to measure an intensity of the light at one or more desired wavelengths of the light after it passes through the liquid hydrocarbon stream in the flow chamber. The intensity measurement of the at least one photosensitive detector array is used to quantify the undissolved water content of the liquid hydrocarbon stream.

In another aspect, the at least one photosensitive detector array is configured to measure the intensity of the transmitted light across an array of detecting elements to provide a 1-dimensional output of light intensity across the width of the flow chamber.

In another aspect, the at least one photosensitive detector array is configured to measure the specific wavelength of transmitted light via the inclusion of at least one bandpass filter located between the flow chamber and the at least one photosensitive detector array.

In another aspect, the at least one photosensitive detector array is configured to measure only the specific wavelengths of the transmitted light through using a prism.

In another aspect, the at least one photosensitive detector array is configured to measure the intensity of light at a wavelength of approximately 1450 nm. In a further aspect, the at least one photosensitive detector array is further configured to measure the intensity of light at a wavelength of approximately 700 nm.

In a second aspect, a spectrophotometric system for quantifying water content in a liquid hydrocarbon stream is provided, where the system includes a first light source that produces a first light having a wavelength of approximately 700 nm, and a second light source that produces a second light having a wavelength of approximately 1450 nm. The system also includes a flow chamber containing a sample of the liquid hydrocarbon stream, where the first and second lights are configured to pass through the sample of the liquid hydrocarbon stream. The system also includes first and second photosensitive detector arrays. The first photosensitive detector array is configured to measure an intensity of the first light at the wavelength of approximately 700 nm after passing through the liquid hydrocarbon stream in the flow chamber and the second photosensitive detector array is configured to measure an intensity of the second light at the wavelength of approximately 1450 nm after passing through the liquid hydrocarbon stream in the flow chamber. The differential between the measurement of the first photosensitive detector array and the measurement of the second photosensitive detector array enable the system to quantify a water content in the liquid hydrocarbon stream.

In another aspect, the first and second lights are generated by a full-spectrum light.

In another aspect, the sources of each of the first and second light is a laser.

In another aspect, the first and second lights are each spread into a 2D sheet.

In another aspect, the thickness of the sample of the liquid hydrocarbon stream in the flow chamber is larger than a diameter of the largest water droplet in the sample of the liquid hydrocarbon stream.

In another aspect, both the first photosensitive detector array and the second photosensitive detector array have proportional outputs relative to the amount of light they absorb.

In another aspect, the water content of the liquid hydrocarbon stream is measured in both the size and number of the water droplets.

In a third aspect, a method for quantifying water content in a liquid hydrocarbon stream is provided. In the method, a first light is directed into a sample of the liquid hydrocarbon stream contained within a flow chamber, wherein the first light has a wavelength of approximately 700 nm. A second light is directed into the sample of the liquid hydrocarbon stream contained within the flow chamber, wherein the second light has a wavelength of approximately 1450 nm. An intensity of the first light at the wavelength of approximately 700 nm is then measured with a first photosensitive detector array after passing through the liquid hydrocarbon stream. An intensity of the second light at the wavelength of approximately 1450 nm is then measured with a second photosensitive detector array after passing through the liquid hydrocarbon stream. A water content in the liquid hydrocarbon stream is then quantified based on the differential between the measurement of the first photosensitive detector array and the measurement of the second photosensitive detector array.

In another aspect, the first and second lights are full-spectrum lights.

In another aspect, a source of each of the first and second lights is a laser. In a further aspect, the first and second lights are each spread into a 2D sheet.

In another aspect, the thickness of the sample of the liquid hydrocarbon stream in the flow chamber is larger than a diameter of the largest water droplet in the sample of the liquid hydrocarbon stream.

In another aspect, both the first photosensitive detector array and the second photosensitive detector array have proportional outputs relative to the amount of light they absorb.

In another aspect, the water content of the liquid hydrocarbon stream is measured in both the size and number of the water droplets.

### BRIEF DESCRIPTION OF THE DRAWING FIGURES

FIG. 1 shows an exemplary absorption spectra for water and various hydrocarbon components in accordance with one or more embodiments.

FIG. 2 displays a system diagram showing an exemplary spectrophotometric system that utilizes a two wavelength analysis in accordance with one or more embodiments.

FIG. 3 shows visualizations of exemplary results of the spectrophotometric process to enabling discrimination between water and other contaminants in a hydrocarbon flow in accordance with one or more embodiments.

FIG. 4 shows visualizations of exemplary expected complications in assessing water content in a hydrocarbon flow in accordance with one or more embodiments.

FIGS. 5A-5B shows a system diagram showing another exemplary spectrophotometric system in perspective (FIG. 5A) and cross-sectional (FIG. 5B) views in accordance with one or more embodiments.

FIG. 6 displays a flow diagram of an exemplary method for quantifying water content in a liquid hydrocarbon stream using a spectrophotometric system of the present application in accordance with or more embodiments.

### DETAILED DESCRIPTION OF CERTAIN EMBODIMENTS

By way of overview and introduction, the present application discloses systems and methods for spectrophotometric measurement of water content in liquid hydrocarbon streams. In one or more embodiments, the present systems utilize water droplet counting to measure the water content in the hydrocarbons stream. Specifically, in one or more embodiments, the present application discloses systems and methods of utilizing a spectrophotometric apparatus to quantify the free water content in a liquid hydrocarbon flow by quantifying the number and size of water droplets flowing through a flow channel.

Conventionally, there are some detectors that utilize absorption to measure the water cut. The general working principle involves comparing the difference in absorption between the sample and a known water-free sample and then dividing that by the absorption of a water only sample (corrected for any absorption of the hydrocarbon sample). This yields a relative absorption, which is then equal to the water cut percentage. The formula governing this measurement is described below:

$$Water Cut \% = 100 \% * \frac{\begin{matrix}light absorption of sample - \\ light absorption of hydrocarbon control\end{matrix}}{\begin{matrix}light absorption of water control - \\ light absorption of hydrocarbon control\end{matrix}}$$

While this type of measurement works reasonably well in providing an approximate measurement in highly mixed streams, it provides inconsistent results for hydrocarbon streams having low levels of water contamination. For example, these existing detectors can provide a lower limit of sensitivity of, at best, 2% (with an error of 2%), and thus they are not reliable for measuring hydrocarbon streams with very low water-cut levels (i.e., water-cut levels of 1% or lower).

In biological analyses, flow cytometry is a process that is used to count cells as they pass through a narrow channel and interact with a light source (generally a laser beam), the interaction of which is captured by a sensor and processed to count thousands of cells per second. The systems and methods of the present application are somewhat similar in concept, except that the present system and method count and quantifies the size of water droplets in hydrocarbon streams in order to measure the free water content of these flows.

The present systems and methods can provide live data on the relative quantity of free water in a hydrocarbon stream, particularly in condensate hydrocarbon streams which have very low water concentrations and operate under high flow rate, temperature, and pressure. For instance, in one or more embodiments, a liquid hydrocarbon stream has a pressure of approximately 200-400 psig. In one or more embodiments, the liquid hydrocarbon stream has a temperature of approximately 120-180° F. (˜49-82° C.). In one or more embodiments, the liquid hydrocarbon streams can have a flow rate of approximately 50-60 mbpd. Further, the present systems and methods can be applied to liquid condensate streams as well as other streams to provide accurate and live water content data for low-water hydrocarbon streams.

In one or more embodiments, a spectrophotometric system is provided for quantifying water content in a liquid hydrocarbon stream. The system can include at least one light source, and a flow chamber containing a sample of the liquid hydrocarbon stream. The at least one light source provides a light that is configured to pass through the sample of the liquid hydrocarbon stream. The system can also include at least one photosensitive detector array and optionally at least one filter (e.g., bandpass filter). The photosensitive detector array can measure an intensity of the light at a desired wavelength after it passes through the liquid hydrocarbon stream, which enables the system to quantify a water content in the liquid hydrocarbon stream. In other words, the at least one photosensitive detector array can detect or measure the intensity of the light after it passes through the sample of the liquid hydrocarbon stream in the flow chamber, and in certain embodiments can then match the measured intensity to the absorption spectra if it is filtered to detect the intensity of different wavelengths. The present system can be an online system for measuring the water content of hydrocarbon bulk flow in real time.

In one or more embodiments, the present systems and methods can be configured to accurately measure the water content of any liquid hydrocarbon stream, and especially those streams that have a very small water content, for example approximately 1-100 ppm, 1-200 ppm, 1-300 ppm, 1-400 ppm, 1-500 ppm, 1-600 ppm, 1-700 ppm, 1-800 ppm, 1-900 ppm, 1-1000 ppm or 500-1000 ppm. This is in contrast to existing methods for determining water content of hydrocarbon streams, which have been shown to be unreliable in measurements of hydrocarbon streams with low levels of water.

These and other aspects of the present systems and methods are described in further detail below with reference to the accompanied drawing figures, in which one or more illustrated embodiments and/or arrangements of the apparatus and methods are shown.

The systems and methods of the present application are not limited in any way to the illustrated embodiments and/or arrangements. It should be understood that the systems and methods as shown in the accompanying figures are merely exemplary of the systems and methods of the present application, which can be embodied in various forms as appreciated by one skilled in the art. Therefore, it is to be understood that any structural and functional details disclosed herein are not to be interpreted as limiting the present systems and methods, but rather are provided as a representative embodiment and/or arrangement for teaching one skilled in the art one or more ways to implement the present systems and methods. It should be understood that, as used in the present application, the term “approximately” when used in conjunction with a number refers to any number within 5% of the referenced number, including the referenced number.

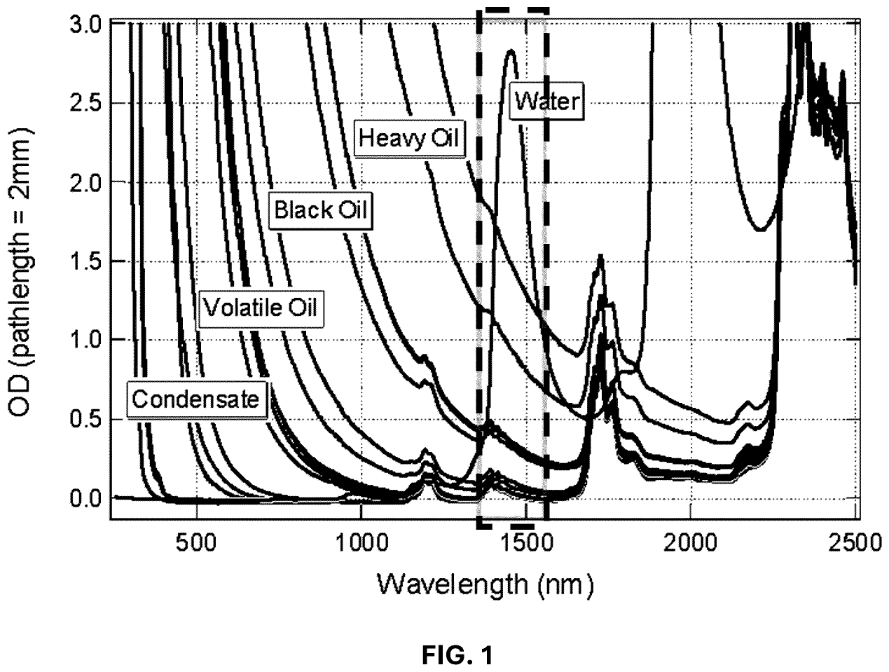

FIG. 1 displays an exemplary absorption spectra for water and hydrocarbons in accordance with one or more embodiments, with a window (dashed box) highlighting water absorption. As shown in the graph of FIG. 1 at the dashed box, among the components often present in hydrocarbon streams—for example water, condensates, volatile oil, black oil, and heavy oil—water displays a distinctive absorption peak at a wavelength of approximately 1450 nm relative to other components in the liquid hydrocarbon stream. Accordingly, the present systems and methods utilize, in part, this distinction to detect and quantify the water content of a liquid hydrocarbon stream.

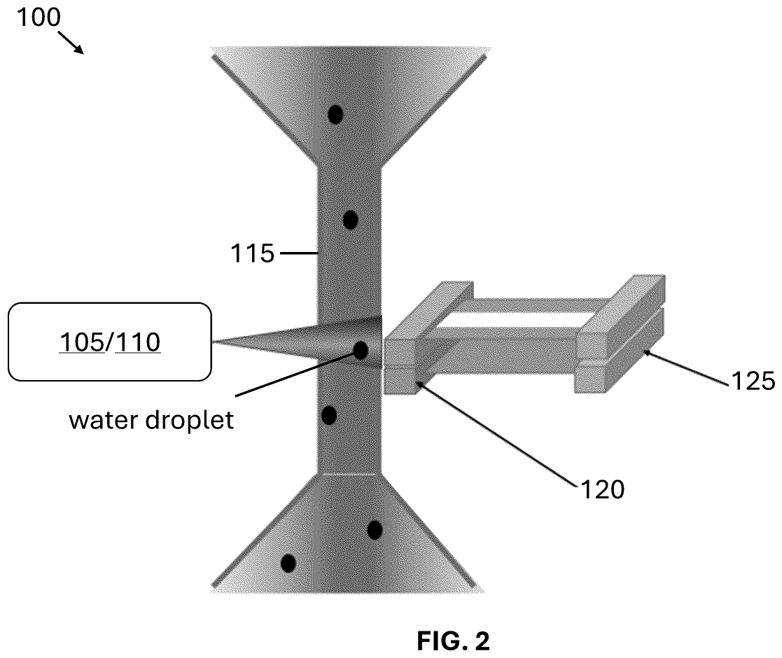

FIG. 2 displays a diagram showing an exemplary spectrophotometric system that utilizes a two wavelength analysis in accordance with one or more embodiments. With reference now to FIG. 2, in one or more embodiments, the spectrophotometric system 100 includes a first light source 105. In certain embodiments, the system 100 further includes a second light source 110 and the light produced by source 105 has a different wavelength than the light produced by source 110. In one or more embodiments, one or both of the light sources can be lasers. In one or more embodiments, the lights produced by either or both of the light sources 105 and 110 can be white lights or full spectrum lights. While in certain embodiments the use of lasers gives more opportunity for control of the relative light intensities, a white light can instead be used as long as high-quality, narrow filters (e.g., narrow bandpass filters) are utilized and the wavelengths of the lights produced by light sources 105 and 110 are sufficiently separated through a prism or other mechanism. If a prism is used with laser light, filters may not be needed as the prism splits the light to different angles based on the wavelength. However, both a prism and a filter may be preferred if a white light is utilized, to avoid noise in the signals (e.g. 1300 nm-1600 nm wavelengths also being captured by the detector rather than just 1450 nm [+/−20 nm]). In one or more embodiments, the filtering can be done prior to the light being exposed to sensors for each bandwidth, or alternatively the sensor arrays can have a grid of differentially sensitive pixels (filtered or sensitivity-based) that enable the reconstruction of a multi-wavelength image.

In one or more embodiments, one or both of the lights produced by the first and second light sources can be spread into a two-dimensional (2D) sheet. In one or more embodiments, the two lights can have target wavelengths related to the specific wavelengths being interrogated. For instance, in one or more embodiments, the light source 105 can produce a light having a wavelength of approximately 1450 nm. In at least one embodiment, the light source 105 can produce a light have a wavelength in the range of approximately 1400-1550 nm. Accordingly, the first light source 105 generally produces a light having wavelength where water exhibits greater absorption than the hydrocarbon components of the stream. In one or more embodiments, the light source 110 can produce a light having a wavelength of approximately 700 nm. In one or more embodiments, the second light source 110 produce a light having a wavelength outside of the approximately 1400-1550 nm range where the hydrocarbon components of the stream have the lowest absorption possible so long as the water absorption is similar to or less than the hydrocarbon absorption at that wavelength.

In one or more embodiments, the system 100 further comprises a flow chamber 115. The flow chamber 115 is configured to hold a sample of a liquid hydrocarbon stream to be analyzed. The sample of the liquid hydrocarbon stream can be fed into the flow chamber 115 in various ways from an existing liquid hydrocarbon flow. For example, an open loop sampler can be used that drains a small amount of fluid from a bulk flow of liquid hydrocarbons to be analyzed and the drain amount can later be disposed of or collected for further processing. Alternatively, a closed loop sampler can be employed to collected the sample of a liquid hydrocarbon flow. The closed loop sampler can utilize active or passive pressure differentials to pull a sample from bulk flow and later return it to the bulk hydrocarbon stream downstream of the sampling point. While, in certain embodiments, closed loop systems are preferred, an open loop system can sometimes be easier to employ, especially in implementations in which accurate measurements can be achieved without requiring significant volumes of a sample to be pulled from the bulk flow. In one or more embodiments, a flow meter (not shown) can be operatively connected to the flow chamber 115 to accurately capture the volume of fluid passing through the flow chamber 115 and to later calculate the relative proportion of free-water in the hydrocarbon sample.

In one or more embodiments, the flow chamber 115 can comprise input windows that allow light from the light sources 105 and 110 to pass through to reach the liquid hydrocarbon stream and additional output windows that allow the light that passes through the liquid hydrocarbon stream to exit the flow chamber 115. The system 100 can further comprise one or more filters 120 (e.g., bandpass filters) which receive the light after it passes through the liquid hydrocarbon stream in the flow chamber 115. In one or more embodiments, the system 100 includes two bandpass filters. The filters 120 only allow a narrow range of wavelengths of light to pass through, specifically the wavelengths of the lights of their respective light sources 105 and 110 (e.g., in certain embodiments, approximately 1450 nm for a filter corresponding the light source 105 and approximately 700 nm for a filter corresponding to light source 110). In other words, in one or more embodiments, for each light source (105 and 110) there can be at least one corresponding filter 120. As shown in the exemplary embodiment of FIG. 2, the black dot shown in the middle of the lights passing through liquid hydrocarbon stream in the flow chamber 115 represents a water droplet blocking the light of the first light source 105 (e.g., 1450 nm) while the light of the second light source 110 (e.g., 700 nm) passes through. In the illustrated embodiment of FIG. 2, the hydrocarbon sample is functionally interrogated in sequence by the first and second lights 105 and 110. Accordingly, in certain embodiments, offsets in the measurements of the first and second lights must be determined using the corresponding detector arrays. Alternatively, in at least one embodiment, a prism can be utilized such that the both lights 105 and 110 pass through a prism and result in the same ‘image’ being displayed on both detector arrays. Additionally, in at least one embodiment, a prism can be used to split light, optionally with filters to produce a cleaner image.

More generally, as the sample of the liquid hydrocarbon stream, which can include condensates, is routed through the flow chamber 115 and it passes through the flow chamber, any contaminants interfere with one or both of the lights (e.g. lasers) passing through the flow chamber 115, which causes a change in the signal being received by the arrays of detectors. In one or more embodiments, an analysis is then conducted to determine the volume of water passing through the chamber as droplets vs the average flow of hydrocarbons in order to calculate a relative proportion of undissolved water content.

As referenced above, the system 100 further comprises one or more arrays of photosensitive detectors 125. In one or more embodiments, the system 100 includes two arrays of photosensitive detectors. In one or more embodiments, each array of photosensitive detectors can be filtered, prismed, and/or illuminated by a single wavelength of light.

In one or more embodiments, the arrays of photosensitive detectors can be 1-D arrays or 2-D arrays. For example, the system 100 can include a first array of photosensitive detectors for detecting the intensity of a first light (e.g., at a wavelength of 1450 nm) across the 1-D or 2-D array and a second array of photosensitive detectors for detection of the intensity of a second light (e.g., at a wavelength of 700 nm) across each pixel of the array. In one or more embodiments, the differential between the intensity measurements of the first and second arrays of photosensitive detectors allow the system to quantify the water content of the liquid hydrocarbon stream by quantifying the number and size of the water droplets flowing through the flow chamber. In one or more embodiments, the water content of the liquid hydrocarbon stream is measured in both the size and number of the water droplets in the sample.

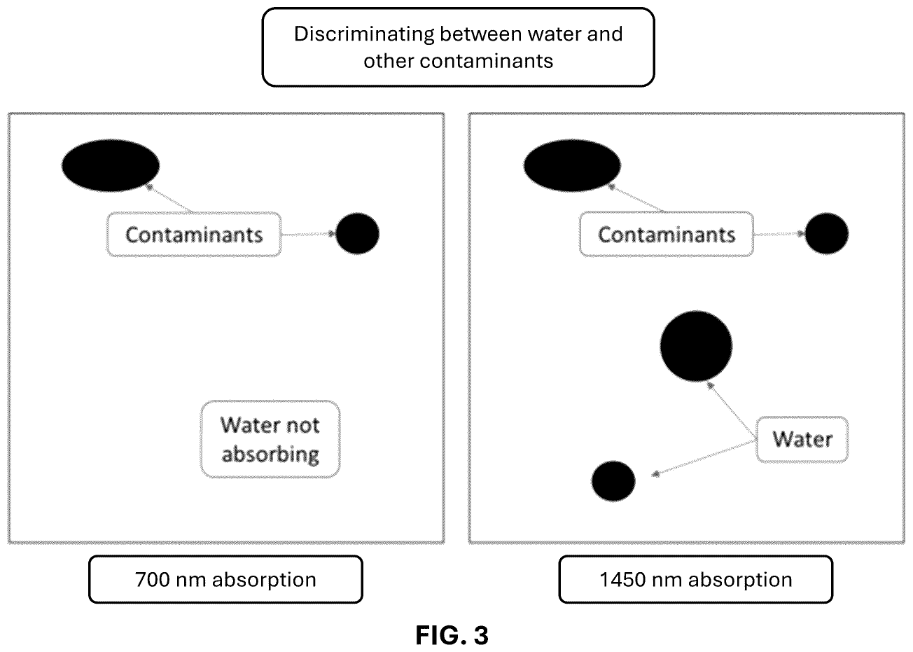

As mentioned above, in one or more embodiments, the arrays of photosensitive detectors can be a 1-D array or a 2-D array. In the case of a 1-D array, the measurements of the 1-D array result in a single line of a picture of the stream (as exemplified FIG. 3) in which the water absorbs the 1450 nm light, for example, and the hydrocarbon does not such that all contaminants show high absorption (or total blockage) of the light, and in the case of the 700 nm light, the water does not absorb the light, so it does not appear in the image. The interrogated rows of the array can then be examined in sequence to reconstruct a 2D image of the flow (e.g., the full picture in FIG. 3), or alternatively a 2-D array of pixels can be used to capture the full image in a single action.

The water content in the hydrocarbon stream can be measured by looking for spots appearing in the 1450 nm spectrum that do not appear in the 700 nm spectrum through differential analysis of the resulting images. This process can have different sequences, such as doing the differential analysis for each row of the array and then showing only the water in an image for calculating water quantities.

In order to convert the differential image showing just water into a water cut percentage of the hydrocarbon stream, the quantity of the water versus the quantity of the oil is calculated by assuming spherical, or elliptical water droplets (based on shape and flow characteristics) and comparing it to the thickness of the fluid sample (e.g., hydrocarbons) being interrogated. In one or more embodiments, the fluid sample being interrogated is held in a rectangular prism (the chamber) roughly the thickness of the largest anticipated water droplet to allow easy flow of the droplets through the chamber, but to avoid issues with focus. In one or more embodiments, an exemplary calculation includes an estimate using only binary data (black/white) in which a sphere takes up approximately 50% of the volume of the smallest cube containing it. Therefore, in one or more embodiments, the water cut can be approximated using the following formula (e.g., for 1450 nm and 700 nm lights):

$$water cut \% = 50 \% * [ ( area absorbed at 1450 nm - area absorbed at 700 nm ) ] / ( area of box ( image ) interrogated ) .$$

In certain embodiments, more complex equations taking advantage of partial absorption can be used to better estimate the size of the droplets and improve the accuracy of the water cut estimation of the hydrocarbon stream.

In one or more embodiments, the differential between the intensity measurements of the first and second arrays of photosensitive detectors allow the system to quantify the presence of other contaminants (e.g., sand, other solid contaminants) in the liquid hydrocarbon stream. The arrays of photosensitive detectors have sufficient resolution to distinguish the size of the contaminants (e.g., water) and enhance the accuracy of the system. More specifically, the interrogated sample of the hydrocarbon stream is thin enough to keep the droplets in good focus and the sizing of the pixels of the detector arrays, after any magnification, are approximately the size of the smallest drop expected or that is significant for the desired precision. We note that, in one or more embodiments, a focusing device can optionally be operatively connected to the photodetector array or located near the photodetector array. The focusing device can augment the detector array's ability to detect small droplets. More specifically, the focusing device can ensure that the light passing through the hydrocarbon flow in the flow chamber is in focus when it reaches the photodetector array.

In one or more embodiments, each array of photosensitive detectors provides a single proportional output signal relative to the amount of light absorbed to get an accurate measurement of free water content. Specifically, for each array of photosensitive detectors, it is not necessary to look at each pixel, because what is important for this analysis is the total number of water droplets in the hydrocarbon sample rather than the positioning of the water drops in the hydrocarbon sample. Instead, a high resolution value can be used to determine the amount of water based on the amount of light absorbed.

In at least one embodiment, where the laser is the source of the lights, spreading the laser beam into a 2D sheet and using an array of detectors (e.g., sensor elements) with proportional output can enable an even more accurate measurement of water content by essentially providing a full image of the liquid hydrocarbon stream over time from which both size and count of drops of water can be determined.

In at least one embodiment, each array of photosensitive detectors can be replaced with a single large detector that can used for detection of the intensity of a light of a particular wavelength. The relative absorption of the large detector based on the specified wavelength of light can still provide information on the water content of the liquid hydrocarbon stream as larger droplets can absorb (block) more light from reaching the detector.

In at least one embodiment, the system can include a single array of photosensitive detectors. In such an embodiment (e.g., RGB cameras), instead of separating the light before the array of detectors, different pixels in the detector can be used to measure the intensity of different wavelengths with placement of the pixels supporting the later reconstruction of an image showing both spectra with high resolution.

In most embodiments of the present application, the photosensitive detectors or arrays of photosensitive detectors are one-dimensional (1D) detectors. In at least one embodiment, at least one of the photosensitive detectors can be a two-dimensional (2D) detector, such as a camera sensor, or an array of 2D detectors. A 2D detector is most appropriate when a laminar flow of the liquid hydrocarbon stream cannot be achieved in the area near the detector (or detector array) in the system. If laminar flow of the liquid hydrocarbon stream in the area near the detector is achieved and the detector can be probed at sufficient frequency or time interval, then a 2D detector is not necessary, as the information collected by a 1D detector array can allow for the reconstruction of a full determination of the contents of the liquid hydrocarbon stream without the need for a 2D detector array. In such an embodiment, in at least one implementation a simplified formula for measuring the water cut (WC) or water content of the hydrocarbon stream can be reduced to: WC %=50%*dark pixels/number of pixels in sensor. The resulting value can then be averaged over a number of readings in order to provide a stable reading for estimating the water cut of the hydrocarbon stream.

In one or more embodiments, the one or more photosensitive detector arrays can be wavelength specific (e.g., for detecting wavelength of 700 nm or 1450 nm). In such, embodiments, the wavelength specific detector arrays can negate the necessity of separate bandpass filters.

As mentioned above, in one or more embodiments, the differential between the intensity measurement of the first photosensitive detector array and the intensity measurement of the second photosensitive detector array allow the system to quantify a water content in the liquid hydrocarbon stream. In embodiments in which the two light sources produce light having a wavelength of approximately 1450 nm and approximately 700 nm, respectively, if the light having a wavelength of approximately 1450 nm passes through the liquid hydrocarbon stream with no absorption or minimal absorption, that indicates that no water content is present in the hydrocarbon stream. As absorption of the 1450 nm light increases (especially as more pixels of the detector array show significant absorption), that indicates that there is greater water content (or other contaminants) present in the liquid hydrocarbon stream.

In one or more embodiments, the light sources are two lasers (e.g., ˜700 nm light and ˜1450 nm light, respectively), with two corresponding bandpass filters, and two corresponding arrays of photosensitive detectors. In certain embodiments, the lasers can be arranged parallel to one another. This type of arrangement can help the system distinguish between the water content in the liquid hydrocarbon stream and other contaminants in the hydrocarbon stream. Specifically, by using a light source and filtered array of detectors in the ˜700 nm range, the light will easily pass through both the light hydrocarbons and the water in the liquid hydrocarbon sample such that any absorption in that spectra could be attributed to other contaminants in the hydrocarbon stream. By using a difference between absorption in the 700 nm range and the 1450 nm range, the system is able to significantly increase the accuracy of water detection in the liquid hydrocarbon sample. Specifically, the use of the 700 nm and 1450 nm lights enable the system to quantify the presence of other contaminants that show absorption in both the 700 nm range and the 1450 nm range, as compared with water, which only shows absorption in the 1450 nm range.

In certain embodiments, the proportion of water volume in the liquid hydrocarbon sample (as well as other contaminants) can be reported to an operator via a computing device operatively connected to the spectrophotometric system. The computing device can be any computing device as is known and understood in the art, and can include some form of user interface, software modules, and hardware, for example, and can communicate with other computing devices via a communication protocol that configures the computing device to send the data to an operator's computing device for further analysis. For example, the computing device can be used to modify process parameters (e.g., via user input) to reduce water cut in the liquid hydrocarbon stream if the water cut reaches or exceeds a user-set threshold. Reduction of water cut in the hydrocarbon stream can be accomplished by, for example, increasing the content of absorbents, changing input ratios, reducing flow rates through separators, or increasing temperature.

In at least one embodiment, the liquid hydrocarbon sample can be evaluated by using two separate light sources at two separate areas in the flow chamber with respective detector arrays arranged at the two separate areas. This type of embodiment can be advantageous if there space constraints or if there is a need to avoid filtering or interference. In this configuration, the use of a bandpass filter for each of the two lights is less important if the detector arrays are physically separated. One potential issue with evaluating the liquid hydrocarbon sample at two separate areas with the respective lights is if the contaminants (water or otherwise) move relative to the flow between the two sampling points. Therefore, in most instances, it is better to evaluate the liquid hydrocarbon sample in one location in the flow chamber and use filters to separate out the relevant wavelengths for detection on the photosensitive detector arrays.

In one or more embodiments, the filters (e.g., bandpass filter) can include mirrored aspects that direct certain wavelengths of light to the respective photosensitive detector arrays. For example, in embodiments with two light sources that produce lights of approximately 1450 nm and 700 nm, the respective filters 120 can include one or more mirrors that direct the 1450 nm light to a first photosensitive detector array and one or more mirrors that direct the 700 nm light to a second photosensitive detector array. The use of mirrors can allow for greater physical separation or distance between the two sets of photosensitive detector arrays. In at least one embodiment, the mirrors can be separate from the filters (e.g., mirrors are upstream of the filters), such that the respective lights, after passing through the flow chamber, are directed to the respective filters by the respective mirrors.

FIG. 3 shows visualizations of exemplary images as a result of the spectrophotometric process to enabling discrimination between water and other contaminants in a liquid hydrocarbon flow in accordance with one or more embodiments. FIG. 3 shows the ideal visualization of the process of enabling discrimination between water and other contaminants in a liquid hydrocarbon flow using the present system in accordance with one or more embodiments (e.g., using a 700 nm light and a 1450 nm light). As shown FIG. 3, in ideal conditions, water does not absorb light at approximately 700 nm wavelength (left image) and thus is not visible in the visualization. In contrast, water shows its greatest absorption of light at approximately 1450 nm wavelength, and thus in the right image of FIG. 3, the water droplets are clearly visible in the visualization. In contrast, other contaminants show absorption at both the 700 nm and 1450 nm wavelengths.

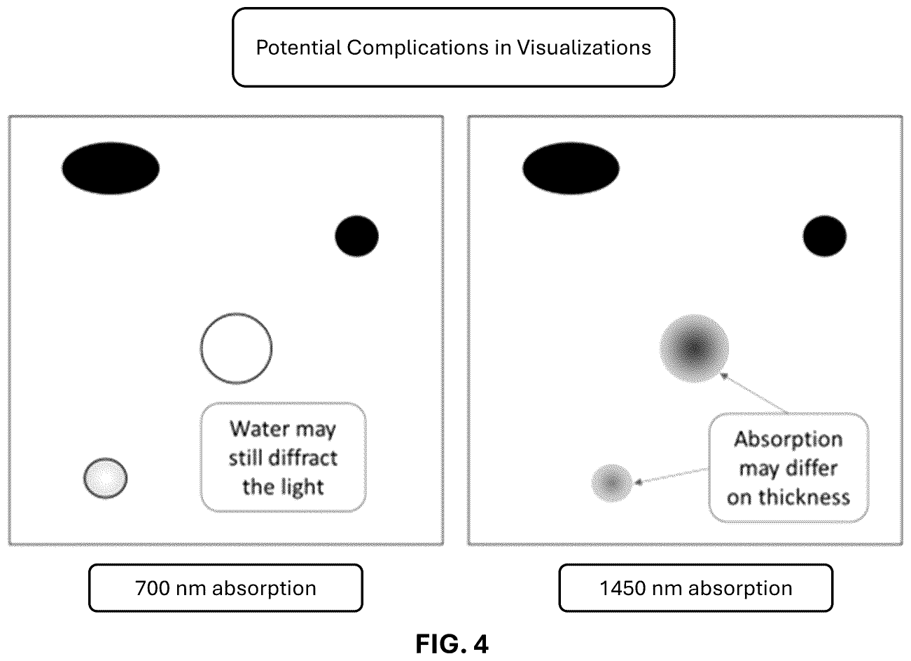

However, the clear results shown in FIG. 3 are not always so easily acquired in every real-world application. FIG. 4 shows expected complications with visual discrimination that need to be accounted for in accordance with one or more embodiments to accurately assess the water content of the hydrocarbon flow in certain instances. For example, as shown in the 700 nm absorption image (left image) of FIG. 4, water droplets can sometimes diffract light in the 700 nm range due to differing optical properties of the hydrocarbons and water, and thereby appear to be indicative of something other than water. These and other complications can affect the measurements of water or other contaminants, depending on their content. For example, in addition to the water diffraction issue, if a water stream is too thick in the liquid hydrocarbon flow, the absorption could change as a result of depth of the contaminant or water droplets and this also needs to be compensated for in certain embodiments.

In certain embodiments, to compensate for the complications with visual discrimination, the detection events can be grouped on a graph with axes matching the two fluorescent wavelengths. In certain implementations, software of a computing device that is operatively connected to the system can assist in appropriately accounting for these complications to allow for more accurate water content measurements. For example, the computing device, executing the software, can be configured to only consider detections that fall within a certain wavelength range for displaying on the graph.

In at least one embodiment, a relatively thin sheet of the liquid hydrocarbon flow can be evaluated in the flow chamber in order to enhance the accuracy of the system. In one or more embodiments, the flow chamber has a depth (i.e., the axis through which light is passing transversely) of approximately 1-1000 μm. In one or more embodiments, water droplet sizes detected in the liquid hydrocarbon flow are no larger than 50% thicker than three standard deviations above the mean thickness for the flow being characterized. However, the thickness of the liquid hydrocarbon flow should be larger than the largest diameter of water drops in the hydrocarbon flow to ensure that the hydrocarbon flow is not selective for the hydrocarbon components of the flow over the water droplets and to ensure the water droplets maintain a relatively consistent shape for volume calculations.

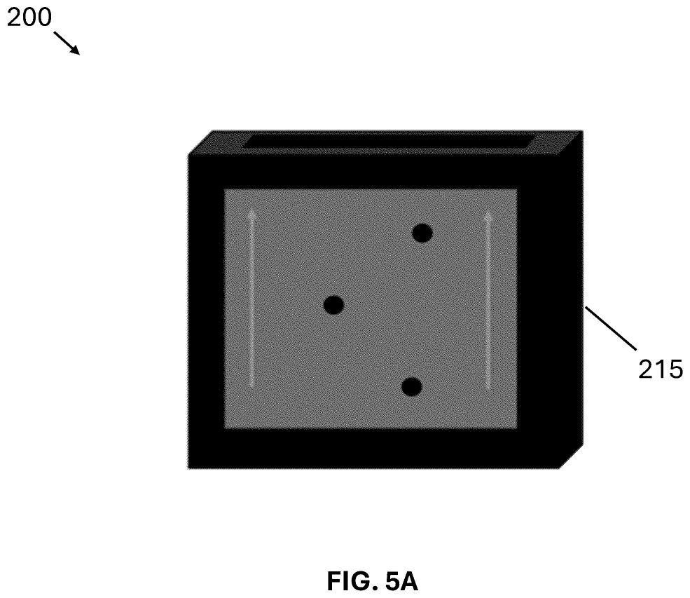

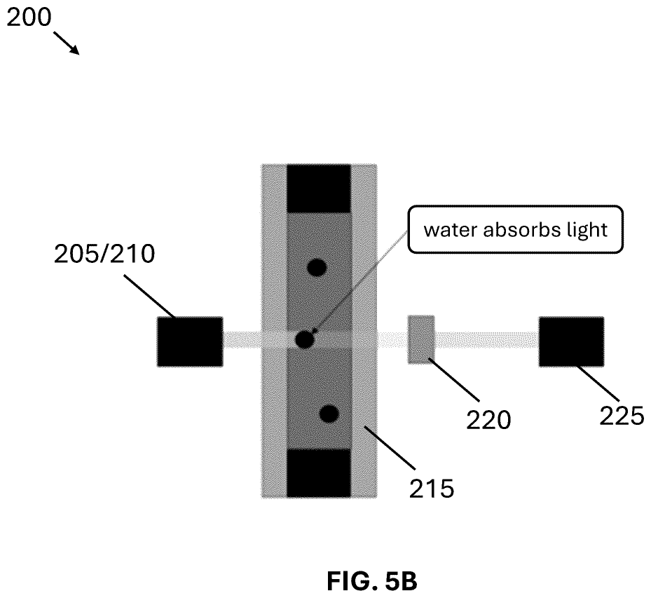

FIGS. 5A-5B shows another exemplary embodiment of the spectrophotometric system having an exemplary flow chamber shown in perspective (FIG. 5A) and cross-sectional (FIG. 5B) views in accordance with one or more embodiments. In this embodiment, the system 200 operates in a substantially similar fashion as the embodiment shown in FIG. 2. The system 200 includes one or more lights sources, for example light sources 205 and 210, that each produce light having two different wavelengths. The system 200 also includes a flow chamber 215 in a rectangular prism configuration, where the flow chamber 215 is configured to convey a sample of the liquid hydrocarbon stream to be analyzed. The system 200 can further comprise one or more filters 220 (e.g., bandpass filters), which receive the light after it passes through the liquid hydrocarbon stream and only allow a narrow range of wavelengths of light to pass through. The system 200 further comprises a one or more arrays of photosensitive detectors 225. In one or more embodiments, the system 200 includes two arrays of photosensitive detectors. As shown in FIGS. 5A-5B, the black dot shown in the middle of the light passing through liquid hydrocarbon stream in the flow chamber 215 represents a water droplet blocking or absorbing the first light (e.g., 1450 nm) while the second light (e.g., 700 nm) passes through to the filter and the detector array.

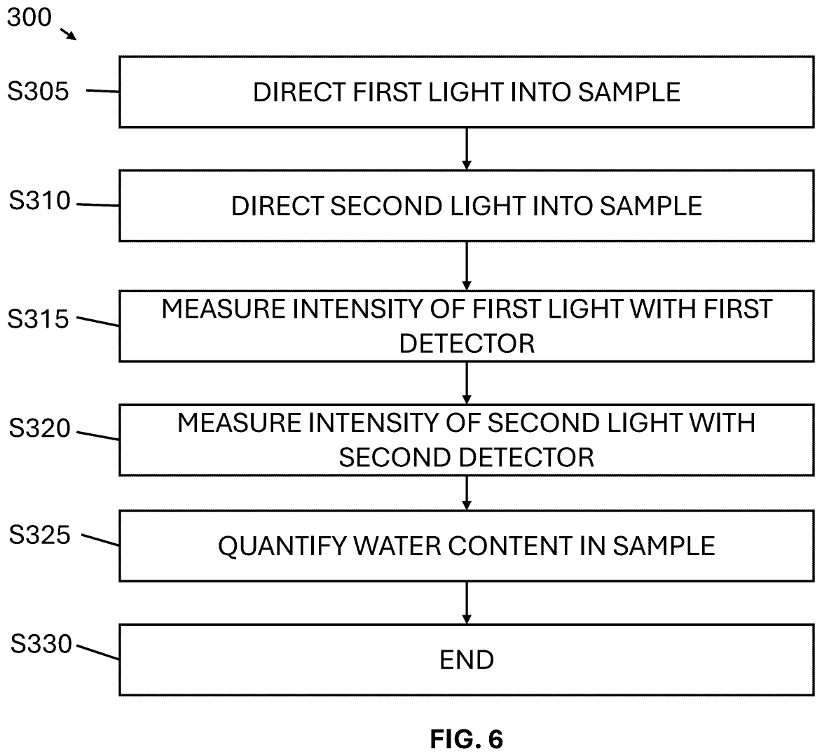

FIG. 6 displays a flow diagram of a method for quantifying water content in a liquid hydrocarbon stream using the spectrophotometric system of the present application in accordance with or more embodiments.

The method 300 begins at step S305 where a first light is directed into a sample of a liquid hydrocarbon stream contained within a flow chamber of the spectrophotometric system. In one or more embodiments, the first light has a wavelength of approximately 700 nm and the first light can optionally passes through a first bandpass filter after passing through the hydrocarbon stream in the flow chamber. The bandpass filter is most useful when other light other than the 700 nm light is leaking into the system. In instances in which only the first light (e.g., 700 nm laser) is passing through the flow chamber and no additional light is leaking in, it is less important to utilize a bandpass filter.

Next, at step S310, a second light is directed into the sample of the liquid hydrocarbon stream contained within the flow chamber, where the second light has a wavelength of approximately 1450 nm, and the second light subsequently passes through a second bandpass filter. In one or more embodiments, the first light and the second light are directed into the sample in the flow chamber at the same time. In at least one embodiment, the first light and the second light are directed into the sample in the flow chamber sequentially, and in such embodiments, there can be a correction for the offset of the timing of the two lights to ensure that the same volume of sample is being compared for each light during the analysis.

At step S315, an intensity of the first light is measured with a first photosensitive detector array, after the first light passes through the liquid hydrocarbon stream and the first bandpass filter. At step S320, an intensity of the second light is measured with a second photosensitive detector array, after the second light passes through the liquid hydrocarbon stream and the second bandpass filter.

It should be understood that in one or more embodiments, steps 305 and 310 can occur in close succession and steps 315 and 320 also occur in close succession. In certain embodiments, steps 305 and 310 occur at the same time and steps 315 and 320 occur at the same time. Alternatively, in embodiments in which there is only one light source, steps 305 and 310 can be combined into a single step, and steps 315 and 320 can be combined into a single step. In certain embodiments in which measurements of the first and second light are made sequentially, step 305 can be followed by 315, and then step 310 can occur, following by step 320. Conversely, in at least one embodiment, steps 305 and 310 can be replaced by a single step of directing a wide spectra light into the sample and then steps 315 and 320 can be combined by reading a 2D image of absorption spectra for the wide spectra light.

With continued reference to FIG. 6, at step S325 a water content in the liquid hydrocarbon stream is quantified based on the difference between the intensity measurement of the first photosensitive detector array and the intensity measurement of the second photosensitive detector array. As part of step S325, in certain embodiments, the content of other contaminants in the liquid hydrocarbon feed can also be quantified based on the intensity measurements. At step S330, the method ends.

The present systems and methods focus on the measurement of droplets of water rather than bulk water, and focuses on counting and measuring the size of these drops to eventually measure the relative % of water with PPM accuracy.

While the present systems and methods utilize certain concepts present in flow cytometry, which is used in biology at a much smaller scale, the present systems and methods provide a unique solution for quantifying contaminants in liquid hydrocarbon streams due to the novel application, specific wavelengths, and array of detectors as compared with the single-cell channel used in the very controlled method of flow cytometry. Similarly, while conventional water cut detectors utilize absorption to distinguish water in bulk flow, none focus on droplet analytics to quantify the water cut at very low concentrations (1% or lower-ppm scales).

Overall, the present systems and methods enable measurement of the water cut of a liquid hydrocarbon bulk flow at ppm levels in an “online” method rather than requiring an offline laboratory method that is inherently prone to errors due to sampling bias as well as difficulty in maintain the sample.

Although much of the foregoing description has been directed to an online spectrophotometric system, the systems and methods disclosed herein can be similarly deployed and/or implemented in scenarios, situations, and settings far beyond the referenced scenarios. It should be further understood that any such implementation and/or deployment is within the scope of the methods described herein.

It is to be further understood that like numerals in the drawings represent like elements through the several figures, and that not all components and/or steps described and illustrated with reference to the figures are required for all embodiments or arrangements. Further, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms ““including,” “comprising,” or “having,” “containing” “involving” and variations thereof herein, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Notably, the figures and examples above are not meant to limit the scope of the present disclosure to a single implementation, as other implementations are possible by way of interchange of some or all the described or illustrated elements. Moreover, where certain elements of the present disclosure can be partially or fully implemented using known components, only those portions of such known components that are necessary for an understanding of the present disclosure are described, and detailed descriptions of other portions of such known components are omitted so as not to obscure the disclosure. In the present specification, an implementation showing a singular component should not necessarily be limited to other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present disclosure encompasses present and future known equivalents to the known components referred to herein by way of illustration.

The foregoing description of the specific implementations will so fully reveal the general nature of the disclosure that others can, by applying knowledge within the skill of the relevant art(s), readily modify and/or adapt for various applications such specific implementations, without undue experimentation, without departing from the general concept of the present disclosure. Such adaptations and modifications are therefore intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s). Itis to be understood that dimensions discussed or shown are drawings are shown accordingly to one example and other dimensions can be used without departing from the disclosure.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

What is claimed is:

1. A spectrophotometric system for quantifying water content in a liquid hydrocarbon stream, comprising:
at least one light source that produces a light having a desired wavelength;
a flow chamber configured to allow passage of a sample of the liquid hydrocarbon stream, wherein the light is configured to pass through the liquid hydrocarbon stream within the chamber; and
at least one photosensitive detector array configured to measure an intensity of the light at one or more desired wavelengths of the light after it passes through the liquid hydrocarbon stream in the flow chamber,
wherein the intensity measurement of the at least one photosensitive detector array is used to quantify the undissolved water content of the liquid hydrocarbon stream.

2. The spectrophotometric system of claim 1, wherein the at least one photosensitive detector array is configured to measure the intensity of the transmitted light across an array of detecting elements to provide a 1-dimensional output of light intensity across the width of the flow chamber.

3. The spectrophotometric system of claim 1, wherein the at least one photosensitive detector array is configured to measure the specific wavelength of transmitted light via the inclusion of at least one bandpass filter located between the flow chamber and the at least one photosensitive detector array.

4. The spectrophotometric system of claim 1, wherein the at least one photosensitive detector array is configured to measure only the specific wavelengths of the transmitted light through using a prism.

5. The spectrophotometric system of claim 1, wherein the at least one photosensitive detector array is configured to measure the intensity of light at a wavelength of approximately 1450 nm.

6. The spectrophotometric system of claim 5, wherein the at least one photosensitive detector array is further configured to measure the intensity of light at a wavelength of approximately 700 nm.

7. A spectrophotometric system for quantifying water content in a liquid hydrocarbon stream, comprising:
a first light source that produces a first light having a wavelength of approximately 700 nm;
a second light source that produces a second light having a wavelength of approximately 1450 nm;
a flow chamber containing a sample of the liquid hydrocarbon stream, wherein the first and second lights are configured to pass through the sample of the liquid hydrocarbon stream;
first and second photosensitive detector arrays, wherein the first photosensitive detector array is configured to measure an intensity of the first light at the wavelength of approximately 700 nm after passing through the liquid hydrocarbon stream in the flow chamber and the second photosensitive detector array is configured to measure an intensity of the second light at the wavelength of approximately 1450 nm after passing through the liquid hydrocarbon stream in the flow chamber;
wherein the differential between the measurement of the first photosensitive detector array and the measurement of the second photosensitive detector array enable the system to quantify a water content in the liquid hydrocarbon stream.

8. The spectrophotometric system of claim 7, wherein the first and second lights are generated by a full-spectrum light.

9. The spectrophotometric system of claim 7, wherein the sources of each of the first and second light is a laser.

10. The spectrophotometric system of claim 7, wherein the first and second lights are each spread into a 2D sheet.

11. The spectrophotometric system of claim 7, wherein the thickness of the sample of the liquid hydrocarbon stream in the flow chamber is larger than a diameter of the largest water droplet in the sample of the liquid hydrocarbon stream.

12. The spectrophotometric system of claim 7, wherein both the first photosensitive detector array and the second photosensitive detector array have proportional outputs relative to the amount of light they absorb.

13. The spectrophotometric system of claim 7, wherein the water content of the liquid hydrocarbon stream is measured in both the size and number of the water droplets.

14. A method for quantifying water content in a liquid hydrocarbon stream, comprising:
directing a first light into a sample of the liquid hydrocarbon stream contained within a flow chamber, wherein the first light has a wavelength of approximately 700 nm;
directing a second light into the sample of the liquid hydrocarbon stream contained within the flow chamber, wherein the second light has a wavelength of approximately 1450 nm;
measuring, with a first photosensitive detector array, an intensity of the first light at the wavelength of approximately 700 nm after passing through the liquid hydrocarbon stream;
measuring, with a second photosensitive detector array, an intensity of the second light at the wavelength of approximately 1450 nm after passing through the liquid hydrocarbon stream; and
quantifying a water content in the liquid hydrocarbon stream based on the differential between the measurement of the first photosensitive detector array and the measurement of the second photosensitive detector array.

15. The method of claim 14, wherein the first and second lights are full-spectrum lights.

16. The method of claim 14, wherein a source of each of the first and second lights is a laser.

17. The method of claim 16, wherein the first and second lights are each spread into a 2D sheet.

18. The method of claim 14, wherein the thickness of the sample of the liquid hydrocarbon stream in the flow chamber is larger than a diameter of the largest water droplet in the sample of the liquid hydrocarbon stream.

19. The method of claim 14, wherein both the first photosensitive detector array and the second photosensitive detector array have proportional outputs relative to the amount of light they absorb.

20. The method of claim 14, wherein the water content of the liquid hydrocarbon stream is measured in both the size and number of the water droplets.
